(19) United States
(12) Patent Application Publication
(10) Pub. No.: US 2020/0239827 A1
(43) Pub. Date: Jul. 30, 2020

(54) ERYTHROCYTE MONITORING DEVICE

(71) Applicant: OLYMPUS CORPORATION, Tokyo (JP)

(72) Inventors: Hiroshi SASAKI, Tokyo (JP); Tatsuya MINAMI, Kanagawa (JP); Yusuke MATSUMOTO, Tokyo (JP)

(73) Assignee: OLYMPUS CORPORATION, Tokyo (JP)

(21) Appl. No.: 16/745,530

(22) Filed: Jan. 17, 2020

(30) Foreign Application Priority Data
Jan. 30, 2019 (JP) 2019-013788

(51) Int. Cl. C12M 1/00; C12M 1/34

(52) U.S. Cl. CPC C12M 41/06; C12M 31/08; C12M 41/32

(57) ABSTRACT

The present invention provides an erythrocyte-culture monitoring device including a light source that radiates monochromatic light onto a culture solution culturing erythrocytes accommodated in a culture container, a first photodetector that detects a light intensity of the monochromatic light transmitted through the culture solution, and a controller that evaluates an amount of hemoglobin in the culture solution based on a temporal change in the light intensity detected by the photodetector.

### CROSS-REFERENCE TO RELATED APPLICATIONS

This application is based on Japanese Patent Application No. 2019-013788, the contents of which are incorporated herein by reference.

### TECHNICAL FIELD

The present invention relates to erythrocyte monitoring devices.

### BACKGROUND ART

In recent years, a technique for differentiating erythrocytes from universal cells, such as iPS cells (induced pluripotent stem cells) and ES cells (embryonic stem cells), has been established (e.g., see Non Patent Literature 1). Such an established technique may possibly be useful in the future for achieving a stable blood transfusion system for erythrocytes.

### CITATION LIST

### Non Patent Literature

### {NPL 1}

“Stem Cell Reports” Dec. 17, 2013, Vol. 1, 499-508, Immortalization of Erythroblasts by c-MYC and BCL-XL Enables Large-Scale Erythrocyte Production from Human Pluripotent Stem Cells

### SUMMARY OF INVENTION

A first aspect of the present invention provides an erythrocyte-culture monitoring device including a light source that radiates monochromatic light onto a culture solution culturing erythrocytes accommodated in a culture container, a first photodetector that detects a light intensity of the monochromatic light transmitted through the culture solution, and a controller that evaluates an amount of hemoglobin in the culture solution based on a temporal change in the light intensity detected by the first photodetector.

### BRIEF DESCRIPTION OF DRAWINGS

FIG. 1 schematically illustrates the configuration of an erythrocyte-culture monitoring device according to a first embodiment of the present invention, as viewed from above.

FIG. 2 schematically illustrates the configuration of the erythrocyte-culture monitoring device in FIG. 1.

FIG. 3 is a graph illustrating molecular absorption coefficients of hemoglobin and water.

FIG. 4 is a graph illustrating an example of absorbance of a culture medium, such as a phenol red solution.

FIG. 5 is a graph illustrating an example of a change (prediction value) in the amount of hemoglobin.

FIG. 6 is a graph illustrating an example of an amount of transmitted light (actually) measured by the erythrocyte-culture monitoring device according to the first embodiment.

FIG. 7 is a flowchart illustrating a process for producing erythrocytes from stem cells.

FIG. 8 is a graph illustrating an absorption spectrum of hemoglobin.

FIG. 9 is a plan view illustrating a light source according to a second modification of the first embodiment.

FIG. 10 is a plan view illustrating a light source according to a fifth modification of the first embodiment.

FIG. 11 is a graph illustrating an example of an amount of transmitted light (actually) measured by the erythrocyte-culture monitoring device according to the fifth modification.

FIG. 12 is a graph illustrating an example of an amount of transmitted light (actually) measured by an erythrocyte-culture monitoring device according to a sixth modification.

FIG. 13 schematically illustrates the configuration of an erythrocyte-culture monitoring device according to a second embodiment of the present invention, as viewed from above.

FIG. 14 schematically illustrates the configuration of the erythrocyte-culture monitoring device in FIG. 13.

FIG. 15 schematically illustrates the configuration of an erythrocyte-culture monitoring device according to a third embodiment of the present invention, as viewed from above.

FIG. 16 schematically illustrates a modification of the erythrocyte-culture monitoring device in FIG. 2.

FIG. 17 schematically illustrates a modification of an erythrocyte-culture monitoring device in FIG. 15.

### DESCRIPTION OF EMBODIMENTS

### First Embodiment

An erythrocyte-culture monitoring device according to a first embodiment of the present invention will be described below with reference to the drawings.

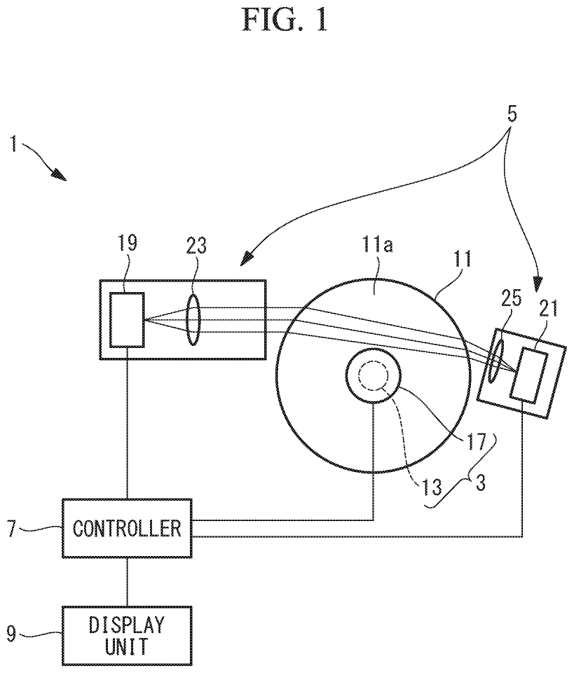

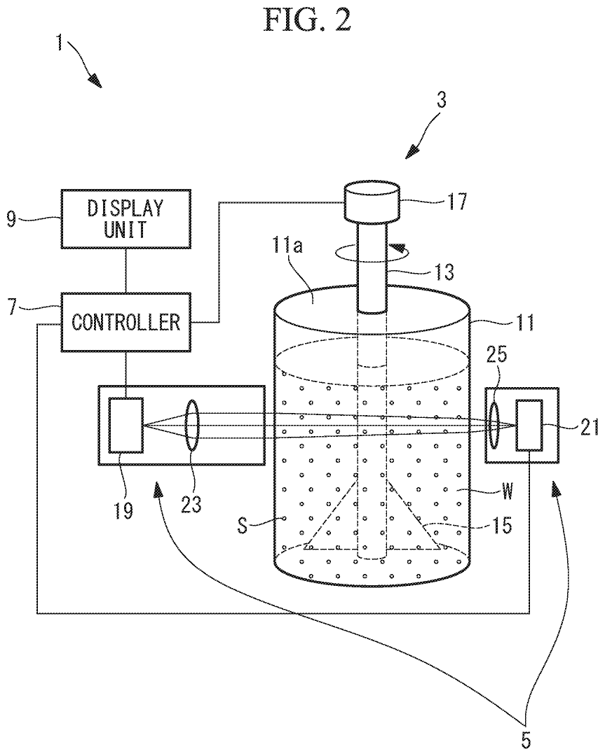

For example, as shown in FIGS. 1 and 2, an erythrocyte-culture monitoring device 1 according to this embodiment includes an stirring mechanism 3 that stirs a culture solution W contained therein together with a culture solution W in a culture container 11, an optical measuring unit 5 that measures the intensity of light transmitted through the culture solution W, a controller 7 that controls the stirring mechanism 3 and the optical measuring unit 5 and that evaluates the amount of hemoglobin in the culture solution W, and a display unit 9 that displays various types of information.

The culture container 11 is, for example, a bioreactor that cultures erythrocytes in a floating state. The culture container 11 has a shape of a closed-end cylinder with a closed upper surface 11a. The culture container 11 is composed of an optically transparent material. The culture solution W used is, for example, a phenol red solution.

The stirring mechanism 3 includes a shaft 13 fitted in the culture container 11 via the upper surface 11a of the culture container 11, a plurality of stirring blades 15 provided on the shaft 13, and a motor 17 that rotates the shaft 13 about its longitudinal axis.

The optical measuring unit 5 includes a light source 19, such as a light emitting diode (LED) or a laser diode (LD), for radiating monochromatic light onto the culture solution W in the culture container 11, and also includes a photodetector (first photodetector) 21, such as a photomultiplier tube, for detecting the amount (i.e., the light intensity) of monochromatic light transmitted through the culture solution W. In FIG. 1, reference sign 23 denotes a collecting lens that collects the monochromatic light emitted from the light source 19 so as to radiate the monochromatic light onto the culture solution W. Reference sign 25 denotes a collecting lens that collects the monochromatic light transmitted through the culture solution W so as to cause the monochromatic light to enter the photodetector 21.

The light source 19 and the photodetector 21 are both disposed outside the culture container 11 so as to substantially face each other with the culture container 11 interposed therebetween in a direction intersecting the depth direction.

The light source 19 radiates monochromatic light toward the culture solution W in the culture container 11 from outside the culture container 11.

The photodetector 21 detects the amount of transmitted light (monochromatic light) output outside the culture container 11 as a result of the monochromatic light radiated onto the culture solution W being transmitted through the culture solution W. This photodetector 21 outputs a detection signal in accordance with the detected amount of transmitted monochromatic light.

For example, the controller 7 includes an interface circuit, a storage unit, such as a hard disk drive, a CPU (central processing unit), and a RAM (random access memory) (none of which is shown).

The interface circuit includes a control substrate for controlling the stirring mechanism 3 and the optical measuring unit 5, and also includes a signal processing substrate that receives the detection signal output from the photodetector 21 and that converts the received detection signal into a light intensity signal.

The storage unit stores various types of programs to be executed by the CPU.

The CPU reads various types of programs stored in the storage unit and executes the following functions. Specifically, the controller 7 performs on/off control of the light source 19 and drives the motor 17 for the stirring mechanism 3. The controller 7 causes the photodetector 21 to temporally detect the amount of monochromatic light transmitted through the culture solution W. Then, the controller 7 evaluates the amount of hemoglobin in the culture solution W based on a temporal change in the amount of transmitted monochromatic light detected by the photodetector 21.

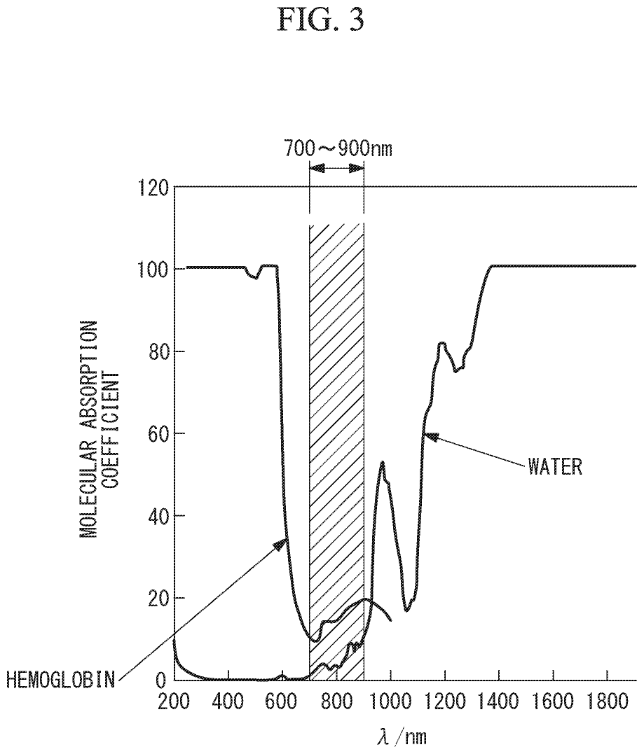

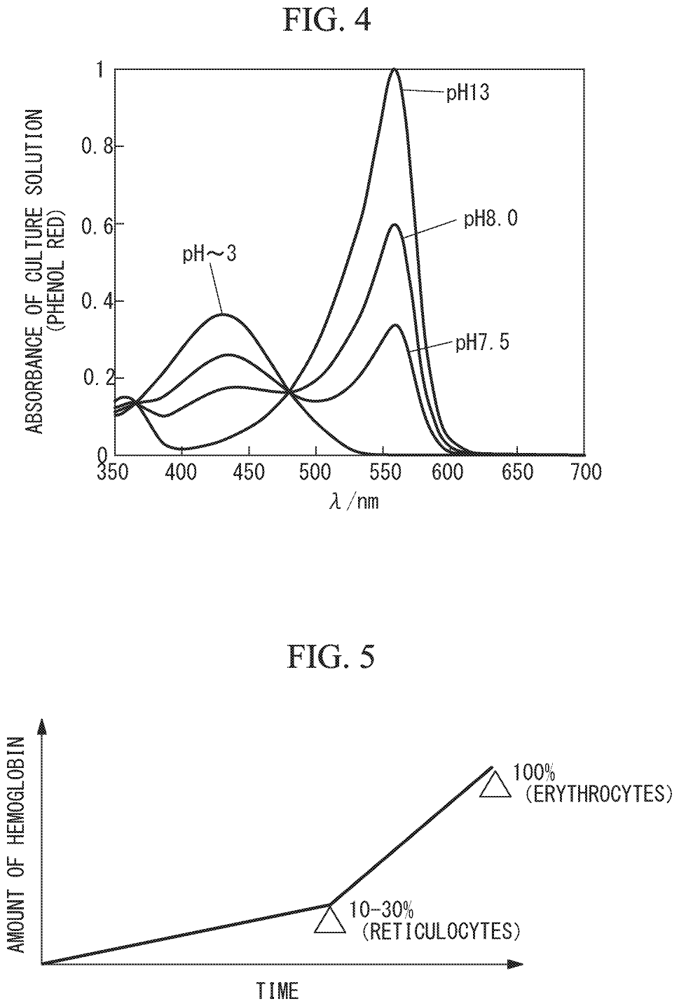

For example, as shown in FIG. 3, there is a water absorption band at a long-wavelength side of 900 nm or longer. In FIG. 3, the abscissa axis indicates a wavelength, and the ordinate axis indicates a molecular absorption coefficient. For example, as shown in FIG. 4, there is an absorption band of the culture solution W, such as a phenol red solution, at a long-wavelength side of 650 nm or shorter. In FIG. 4, the abscissa axis indicates a wavelength, and the ordinate axis indicates the absorbance of the culture solution W. When the hemoglobin absorption coefficient is large and the hemoglobin density is high, the amount of monochromatic light detected by the photodetector 21 decreases, resulting in a lower S/N ratio.

The controller 7 sets, for the light source 19, a wavelength band that excludes the absorption wavelength band of water and the absorption wavelength band of the culture solution W and that covers near-infrared light between 700 nm and 900 nm in which the hemoglobin absorption coefficient is relatively small, as shown in FIG. 3.

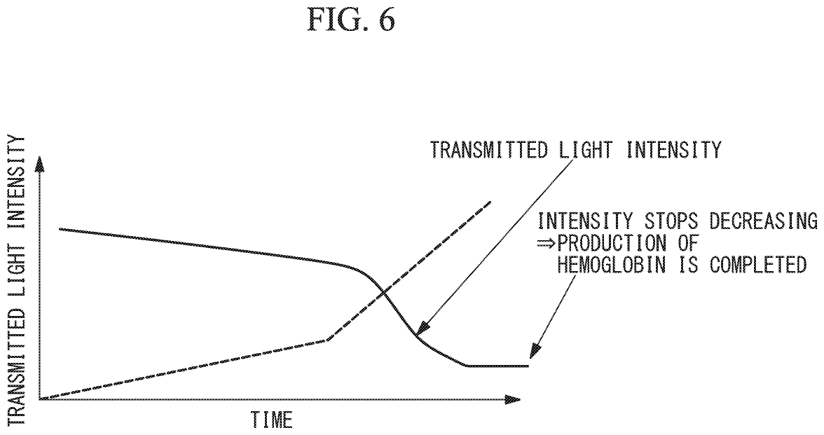

Because hemoglobin is the main substance constituting an erythrocyte, for example, the amount of hemoglobin in the culture solution W increases with increasing number of erythrocytes in the culture solution W, as shown in FIG. 5. In FIG. 5, the abscissa axis indicates elapsed time, and the ordinate axis indicates the amount of hemoglobin in the culture solution W. For example, as shown in FIG. 6, the amount of monochromatic light transmitted through the culture solution W, that is, the light intensity of the monochromatic light detected by the photodetector 21, decreases with increasing amount of hemoglobin in the culture solution W. In FIG. 6, the abscissa axis indicates elapsed time, and the ordinate axis indicates the intensity of transmitted light.

When the light intensity of monochromatic light temporally detected by the photodetector 21, that is, the amount of transmitted light, stops decreasing at a certain value, the controller 7 determines that the production of erythrocytes, that is, the production of hemoglobin, is completed. Then, the controller 7 causes the display unit 9 to display a message indicating that the production of erythrocytes is completed.

Next, the operation of the erythrocyte-culture monitoring device 1 according to this embodiment will be described.

A method for culturing erythrocytes from ES cells S while monitoring the differentiation of the erythrocytes being cultured by using the erythrocyte-culture monitoring device 1 having the above-described configuration will now be described.

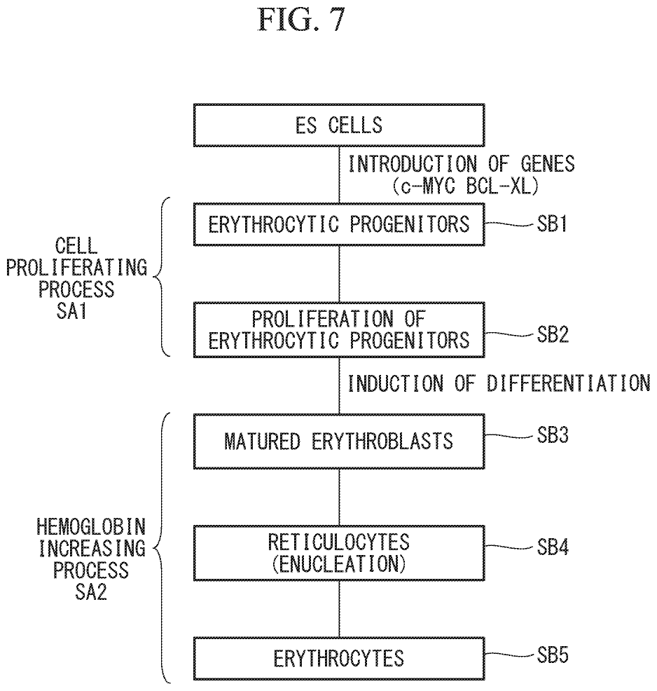

For example, as shown in a flowchart in FIG. 7, the process for culturing erythrocytes from ES cells S is divided into a cell proliferating process SA1 for causing ES cells S serving as a base for erythrocytes to proliferate, and a hemoglobin increasing process SA2 for changing the ES cells S proliferated as a result of the cell proliferating process SA1 into hemoglobin. In the cell proliferating process SA1 and the hemoglobin increasing process SA2, the stirring mechanism 3 is driven by the controller 7 so as to perform the culturing process while stirring the culture solution W in the culture container 11.

The cell proliferating process SA1 involves, for example, producing erythrocytic progenitors by introducing two kinds of genes, such as C-MYC and BCL-XL, to the ES cells S (step SB1) and causing the produced erythrocytic progenitors to proliferate in the culture container 11 containing the culture solution W (step SB2).

The hemoglobin increasing process SA2 involves producing erythroblasts from the erythrocytic progenitors by adding a differentiation-inducing factor to the culture solution W (step SB3), producing reticulocytes from the matured erythroblasts (step SB4), and differentiating erythrocytes from the reticulocytes (step SB5).

In order to monitor the differentiation of the erythrocytes being cultured by using the erythrocyte-culture monitoring device 1 having the above-described configuration, for example, the controller 7 turns on the light source 19 in the hemoglobin increasing process SA2 so as to radiate monochromatic light in a near-infrared wavelength band between 700 nm and 900 nm onto the culture solution W culturing the erythrocytes. Then, the controller 7 controls the photodetector 21 so as to temporally detect the amount of monochromatic light transmitted through the culture solution W.

Subsequently, as shown in FIGS. 5 and 6, since the light intensity of the monochromatic light detected by the photodetector 21 decreases, that is, the amount of transmitted light decreases, as the erythrocytes in the culture solution W proliferate, the controller 7 evaluates the degree of increase in hemoglobin in the culture solution W based on a temporal change in the amount of transmitted light detected by the photodetector 21.

When the amount of transmitted light stops decreasing at a certain value, the controller 7 determines that most of the reticulocytes in the culture solution W have changed to erythrocytes and that the production of erythrocytes, that is, the production of hemoglobin, is completed. Then, the controller 7 causes the display unit 9 to display a message indicating that the production of erythrocytes is completed.

As described above, in the erythrocyte-culture monitoring device 1 according to this embodiment, the controller 7 evaluates the amount of hemoglobin in the culture solution W based on a temporal change in the amount of monochromatic light transmitted through the culture solution W culturing erythrocytes, so that the controller 7 can readily determine whether or not the differentiation of the erythrocytes in the culture solution W is progressing properly. Therefore, the differentiation of the erythrocytes being cultured can be easily monitored.

This embodiment may be modified as follows.

In this embodiment, the light source 19 radiates monochromatic light in a near-infrared wavelength band between 700 nm and 900 nm. As a first modification, in the near-infrared wavelength band between 700 nm and 900 nm, the light source 19 may radiate monochromatic light in a range in which the absorption coefficient of oxygenated hemoglobin (Oxy Hb) and the absorption coefficient of deoxygenated hemoglobin (Deoxy Hb) are substantially equal to each other, such as a near-infrared wavelength band of 805 nm±20 nm.

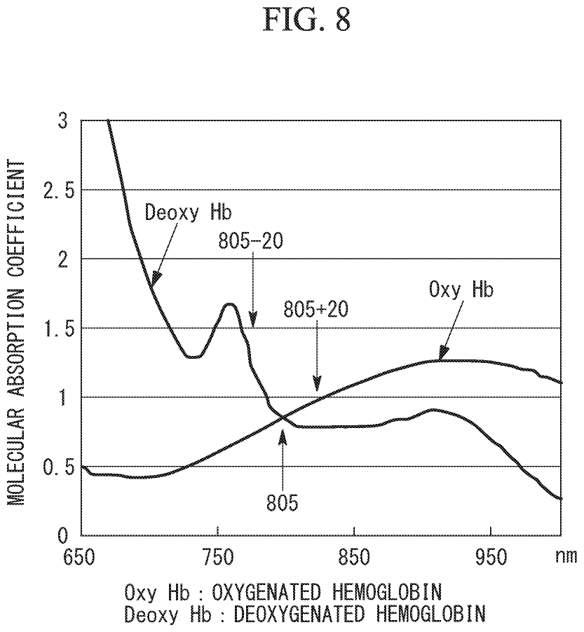

The hemoglobin absorption spectra of oxygenated hemoglobin and deoxygenated hemoglobin differ significantly, as shown in, for example, FIG. 8. In FIG. 8, the abscissa axis indicates a wavelength, and the ordinate axis indicates a molecular absorption coefficient. Therefore, the measurement of the light intensity of monochromatic light becomes unstable due to oxygen concentration, sometimes making it impossible to accurately determine the timing at which the amount of monochromatic light transmitted through the culture solution W becomes saturated.

In contrast, when the wavelength band of near-infrared light to be radiated from the light source 19 is set to the range in which the absorption coefficient of oxygenated hemoglobin and the absorption coefficient of deoxygenated hemoglobin are substantially equal to each other, the amount of transmitted light does not change between oxygenated hemoglobin and deoxygenated hemoglobin. Thus, the timing at which the amount of monochromatic light transmitted through the culture solution W becomes saturated can be accurately determined.

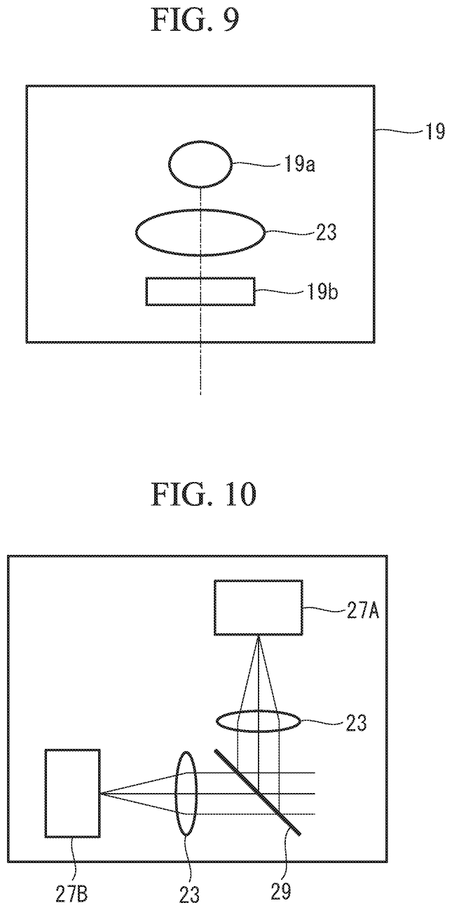

As a second modification, for example, as shown in FIG. 9, the light source 19 may be constituted by a white light source unit 19a, such as a halogen light source, a collecting lens 23 that collects light emitted from the white light source unit 19a, and a bandpass filter 19b that extracts a specific wavelength from the light collected by the collecting lens 23.

According to this modification, since the halogen light source and the bandpass filter are inexpensive, cost reduction can be achieved. The light source 19 constituted by the white light source unit 19a and the bandpass filter 19b has a high degree of freedom for wavelength selection and can thus be applied to various culture solutions W.

As an alternative to the bandpass filter 19b used in this modification, for example, a wavelength-selectable slit, such as a diffraction grating or a monochromator, may be used.

According to this configuration, the degree of freedom for wavelength selection can be further improved.

In this embodiment, the degree of production of erythrocytes, that is, the degree of production of hemoglobin, is evaluated based on the amount of transmitted light. In a third modification, the completion of production of erythrocytes, that is, production of hemoglobin, may be evaluated based on transmittance (T) or absorbance (A) of the monochromatic light in the culture solution W.

If transmittance (T) is to be used, an input light amount (I₀) of monochromatic light entering the culture solution W may be measured before starting the culturing of erythrocytic progenitors by using the culture container 11. Then, the degree of production of erythrocytes may be evaluated based on a ratio, that is, T=I/I₀, between the input light amount (I₀) and an amount of monochromatic light, that is, an output light amount (I), transmitted through the culture solution W and detected by the photodetector 21.

As an alternative to measuring the input light amount (I₀) before starting the culturing of erythrocytic progenitors using the culture container 11, for example, the degree of production of erythrocytes may be evaluated based on a ratio, that is, T=I/I₁, between an amount (I₁) of monochromatic light before being transmitted through the culture solution W and an amount (I) of monochromatic light transmitted through the culture solution W and detected by the photodetector 21.

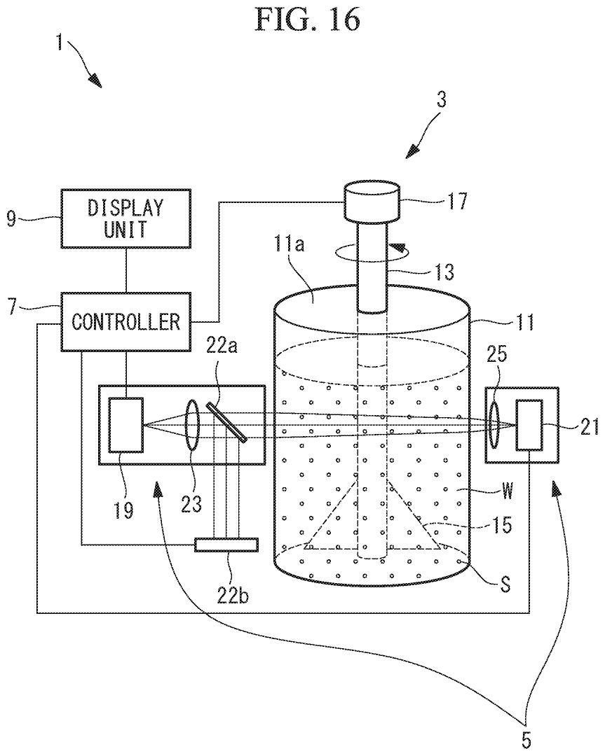

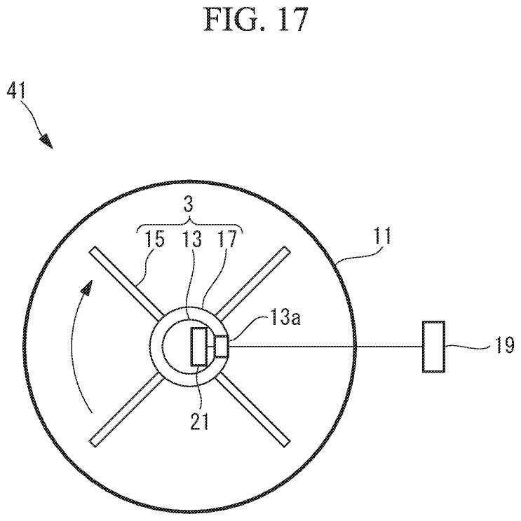

In this case, as shown in FIG. 16, for example, a half mirror 22a that splits the optical path of the monochromatic light emitted from the light source 19 in front of the culture solution W and a photodetector (second photodetector) 22b that detects the amount of monochromatic light whose optical path is split by the half mirror may be provided. By using the photodetector 22b, the amount (I₁) of monochromatic light before being transmitted through the culture solution W may be detected at all times. According to this configuration, the amount of hemoglobin in the culture solution W can be properly evaluated even when there is a change in the input light amount of the monochromatic light entering the culture solution W, that is, the light intensity of the monochromatic light emitted from the light source 19.

If absorbance (A) is to be used, it may be determined that the production of erythrocytes is completed at the timing at which the absorbance has stopped increasing based on A=−log(T).

As a fourth modification, for example, the culturing of erythrocytes may be monitored in a state where the entire erythrocyte-culture monitoring device 1 including the optical measuring unit 5 and the culture container 11 is set in a dark place.

According to this configuration, the transmittance of the monochromatic light transmitted through the culture solution W can be measured accurately without being affected by light from a lighting device, light from a monitor, and external light.

As a fifth modification, the wavelength of monochromatic light whose light intensity is to be measured may be varied between the cell proliferating process SA1 and the hemoglobin increasing process SA2.

In this case, for example, as shown in FIG. 10, a light source unit 27A that emits 630 nm monochromatic light, a light source unit 27B that emits 800 nm monochromatic light, and a dichroic mirror 29 that combines the beams of monochromatic light emitted from these light source units 27A and 27B may be used in place of the light source 19.

In the cell proliferating process SA1, a wavelength between 600 nm and 650 nm, for example, 630 nm emitted from the light source unit 27A, may be used. A wavelength between 600 nm and 650 nm tends to scatter more easily than a wavelength between 700 nm and 900 nm due to being shorter, and is advantageous in having higher sensitivity to a change in cell density. As shown in FIG. 4, a wavelength between 600 nm and 650 nm is also advantageous in that the effect of absorption by the culture solution W is relatively small.

On the other hand, in the hemoglobin increasing process SA2, a wavelength between 700 nm and 900 nm, for example, 800 nm emitted from the light source unit 27B, may be used. As shown in FIG. 3, a wavelength between 700 nm and 900 nm has higher transmittance of hemoglobin than a wavelength between 600 nm and 650 nm, so that an S/N ratio for measuring the amount of transmitted light can be ensured. A wavelength between 700 nm and 900 nm is advantageous in being less affected by scattering of cells S due to being longer.

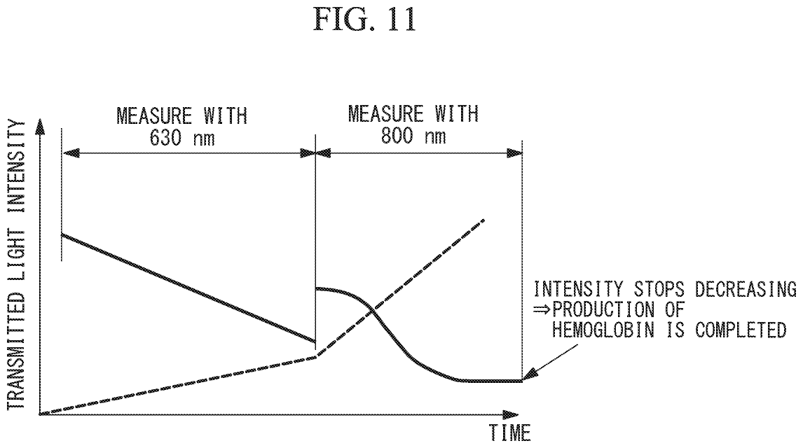

Therefore, for example, as shown in FIG. 11, the wavelength of the monochromatic light is varied between the cell proliferating process SA1 and the hemoglobin increasing process SA2, so that the degree of progression of culturing can be accurately ascertained for each process. In particular, the degree of proliferation of cells S in the cell proliferating process SA1 can be detected with high accuracy. In FIG. 11, the abscissa axis indicates elapsed time, and the ordinate axis indicates the intensity of transmitted light.

This modification may be modified as follows.

For example, the measurement in the cell proliferating process SA1 and the measurement in the hemoglobin increasing process SA2 may both be performed by using both a wavelength suitable for measurement in the cell proliferating process SA1, such as 630 nm, and a wavelength suitable for measurement in the hemoglobin increasing process SA2, such as 800 nm. Specifically, the measurement in the cell proliferating process SA1 may be performed using two wavelengths, namely, 630 nm and 800 nm, and the measurement in the hemoglobin increasing process SA2 may be performed using two wavelengths, namely, 630 nm and 800 nm.

In this case, for example, two photodetectors 21 may be provided, and 630 nm monochromatic light emitted from one light source unit 27A and 800 nm monochromatic light emitted from the other light source unit 27B may be combined by using, for example, a dichroic mirror, and may be radiated onto the culture solution W. Then, the monochromatic light of each wavelength transmitted through the culture solution W may be split for each wavelength by using, for example, a dichroic mirror, and the split beams of monochromatic light of the two wavelengths may be detected simultaneously by the two photodetectors 21. The 630 nm monochromatic light and the 800 nm monochromatic light may be used in a switching manner in the cell proliferating process SA1 and the hemoglobin increasing process SA2.

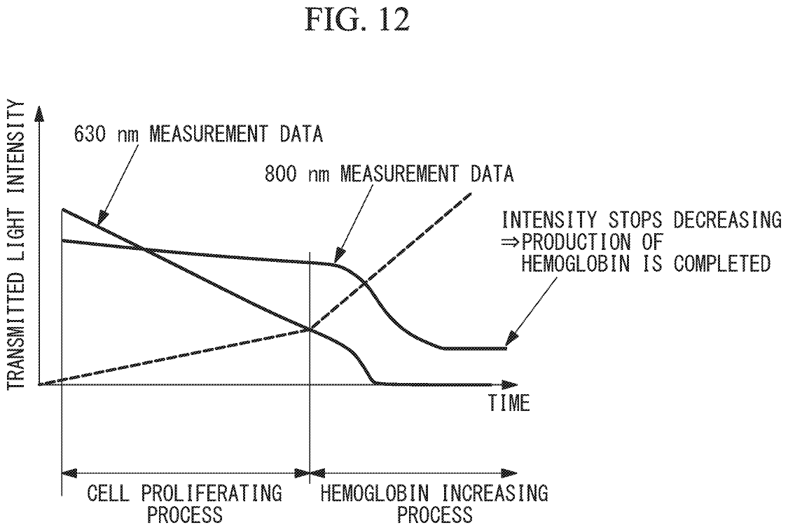

In this case, for example, as shown in FIG. 12, in the cell proliferating process SA1, it is not possible to detect a state of an increase in the number of cells since there is no effect of scattering of the cells S, that is, since the absorption coefficient of the cells S is small, when the wavelength is 800 nm, whereas it is possible to detect a state of an increase in the number of cells S since there is an effect of scattering of the cells S when the wavelength is 630 nm. In FIG. 12, the abscissa axis indicates elapsed time, and the ordinate axis indicates the intensity of transmitted light.

Next, in the hemoglobin increasing process SA2, it is not possible to detect an increase in hemoglobin at a high S/N ratio since the intensity of transmitted light is too low due to an extremely high hemoglobin absorption coefficient when the wavelength is 630 nm, whereas it is possible to accurately detect an increase in hemoglobin since the hemoglobin absorption coefficient is moderately low when the wavelength is 800 nm.

Therefore, according to this modification, the data for both wavelengths are measured even when the user is not able to ascertain the timing for switching from the cell proliferating process SA1 to the hemoglobin increasing process SA2. This is advantageous in that data analysis is possible after the measurement.

### Second Embodiment

Next, an erythrocyte-culture monitoring device according to a second embodiment of the present invention will be described.

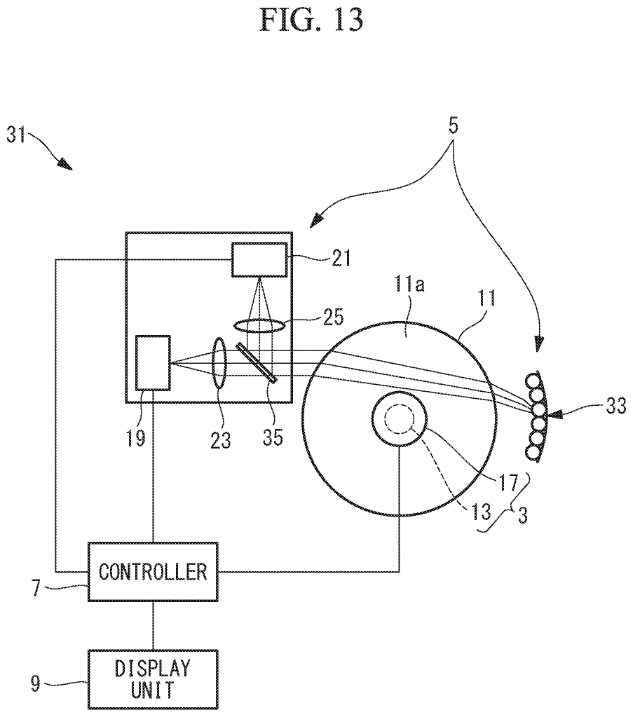

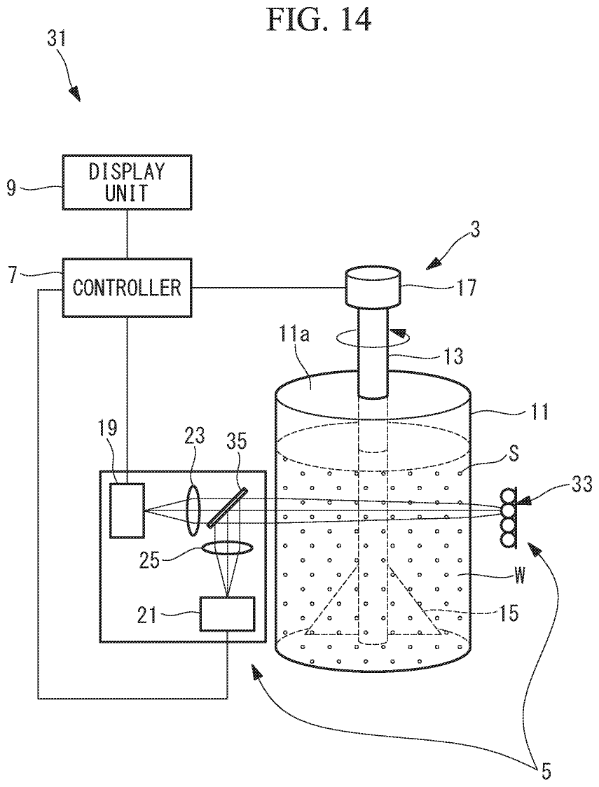

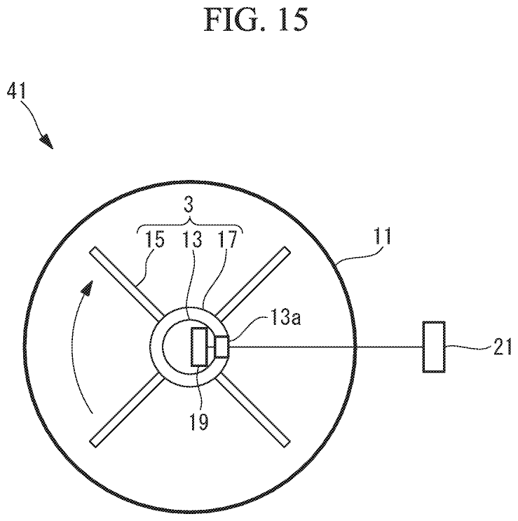

For example, as shown in FIGS. 13 and 14, an erythrocyte-culture monitoring device 31 according to this embodiment is different from that in the first embodiment in that the optical measuring unit 5 includes a retroreflector member 33 that causes the monochromatic light transmitted through the culture solution W to return toward the light source 19 and a half mirror (optical-path splitting member) 35 that splits the optical path of the monochromatic light returned by the retroreflector member 33.

Components identical to those of the erythrocyte-culture monitoring device 1 according to the first embodiment will be given the same reference signs, and descriptions thereof will be omitted.

The retroreflector member 33 is disposed outside the culture container 11 so as to substantially face the light source 19 with the culture container 11 interposed between the light source 19 and the retroreflector member 33 in a direction intersecting the depth direction. Monochromatic light radiated onto the culture solution W from the light source 19 is transmitted through the culture solution W so that the transmitted light (monochromatic light) output outside the culture container 11 enters the retroreflector member 33. The retroreflector member 33 can reflect the input monochromatic light in a direction opposite to the input direction, and can cause the monochromatic light to return toward the culture container 11 via the same optical path as the optical path of the input monochromatic light.

The position and angle of the retroreflector member 33 may be set in a freely chosen manner. The retroreflector member 33 may be attached to, for example, a stand or a wall (not shown), or may be attached to a side surface of the culture container 11. The installation position and installation angle of the retroreflector member 33 may be any position and any angle that do not cause deviation of the reflected monochromatic light.

For example, the half mirror 35 is disposed in the optical path of the monochromatic light between the light source 19 and the culture container 11. The half mirror 35 reflects the monochromatic light, reflected by the retroreflector member 33 and subsequently transmitted again through the culture container 11, toward the photodetector 21 so as to guide the monochromatic light toward the photodetector 21.

Next, the operation of the erythrocyte-culture monitoring device 31 according to this embodiment will be described.

If the differentiation of erythrocytes being cultured is to be monitored by using the erythrocyte-culture monitoring device 31 having the above-described configuration, monochromatic light emitted from the light source 19 is transmitted through the half mirror 35 and is subsequently radiated onto the culture solution W in the culture container 11.

The monochromatic light transmitted through the culture solution W is reflected by the retroreflector member 33 and returns toward the culture container 11 via the same optical path as the optical path of the monochromatic light entering the retroreflector member 33. Then, the monochromatic light is transmitted again through the culture solution W in the culture container 11 and is subsequently reflected by the half mirror 35 so as to enter the photodetector 21. Accordingly, the amount of monochromatic light transmitted through the culture solution W is detected by the photodetector 21. Since an evaluation of the amount of hemoglobin in the culture solution W by the controller 7 is the same as that in the first embodiment, a description thereof will be omitted.

With the erythrocyte-culture monitoring device 31 according to this embodiment, the retroreflector member 33 and the half mirror 35 can cause the monochromatic light transmitted through the culture solution W to enter the photodetector 21, regardless of the surface shape, size, and position of the culture container 11. Therefore, the light intensity of monochromatic light transmitted through the culture solution W can be accurately measured even when various types of culture containers 11 are used. The user can be prompted to replace the culture solution W at an appropriate timing, regardless of the surface shape, size, and position of the culture container 11.

### Third Embodiment

Next, an erythrocyte-culture monitoring device according to a third embodiment of the present invention will be described.

For example, as shown in FIG. 15, an erythrocyte-culture monitoring device 41 according to this embodiment is different from that in the first embodiment in that the light source 19 is disposed inside the culture container 11 and the photodetector 21 is disposed outside the culture container 11.

Components identical to those of the erythrocyte-culture monitoring device 1 according to the first embodiment will be given the same reference signs, and descriptions thereof will be omitted.

In the erythrocyte-culture monitoring device 41 according to this embodiment, the shaft 13 of the stirring mechanism 3 is formed of a hollow cylindrical member. The shaft 13 has a parallel-plate-like optical window 13a composed of an optically transparent material. The optical window 13a is disposed so as to be insertable into the optical path of the monochromatic light connecting the light source 19 and the photodetector 21 as the shaft 13 rotates about an optical axis.

The light source 19 is accommodated in the shaft 13 and is disposed facing the photodetector 21. The light source 19 does not rotate about the optical axis of the shaft 13 and outputs monochromatic light toward the photodetector 21 from inside the shaft 13 via the optical window 13a of the shaft 13 inserted in the optical path of the monochromatic light connecting the light source 19 and the photodetector 21.

With the erythrocyte-culture monitoring device 41 according to this embodiment, the monochromatic light emitted from the light source 19 is radiated onto the culture solution W in the culture container 11 at the timing at which the optical window 13a is inserted in the optical path of the monochromatic light connecting the light source 19 and the photodetector 21 as the shaft 13 rotates, and the monochromatic light transmitted through the culture solution W is detected by the photodetector 21.

In this case, since the light source 19 is disposed inside the culture container 11, space can be ensured outside the culture container 11. Because the light source 19 is not rotated, a circuit connected to the light source 19 does not have to be complex. With the light source 19 being accommodated in the shaft 13, the light source 19 is prevented from interfering with the flow of the culture solution W and the cells S.

As an alternative to this embodiment in which the light source 19 is disposed inside the culture container 11 and the photodetector 21 is disposed outside the culture container 11, as shown in FIG. 17, the photodetector 21 may be disposed inside the culture container 11 and the light source 19 may be disposed outside the culture container 11.

In this case, the photodetector 21 may be accommodated in the shaft 13 and may be disposed facing the light source 19. The photodetector 21 does not rotate about the optical axis of the shaft 13, and detects the amount of monochromatic light entering the shaft 13 via the optical window 13a at the timing at which the optical window 13a of the shaft 13 is inserted in the optical path of the monochromatic light connecting the light source 19 and the photodetector 21.

As described above, the present invention involves radiating monochromatic light onto a culture solution culturing erythrocytes, detecting the light intensity of the monochromatic light transmitted through the culture solution, and evaluating the amount of hemoglobin in the culture solution based on a temporal change in the transmitted light intensity. In addition, the present invention may include an imaging optical system constituted by an optical system and an imaging element for acquiring a culture-solution image in the culture solution.

As shown in FIG. 7, enucleation of erythrocytes occurs as hemoglobin increases, causing the erythrocytes to change in shape.

Based on a change in the image acquired by the imaging optical system, it is possible to detect that the erythrocytes have enucleated. It is possible to monitor that hemoglobin is properly produced not only from the amount of hemoglobin but also from a change in the shape of the erythrocytes.

Although the embodiments of the present invention have been described above in detail with reference to the drawings, specific configurations are not limited to these embodiments, and design alterations are also included in the present invention so long as they do not depart from the scope thereof. For example, the present invention is not limited to each of the above embodiments and the modifications thereof, and may be applied to an embodiment obtained by appropriately combining these embodiments and modifications and is not limited in particular.

In each of the above embodiments, the culture container 11 described has a shape of a closed-end cylinder and is composed of an optically transparent material. Alternatively, the culture container used may have any freely chosen shape, such as a bag shape, a spherical shape, or a box shape. For example, the culture container used may be a disposable bag. The culture container may be composed of any freely chosen material, including a rigid material or a soft material, such as vinyl. The culture container 11 does not have to be entirely transparent. The culture container 11 may partially have a transparent section through which the monochromatic light is transmitted.

A first aspect of the present invention provides an erythrocyte-culture monitoring device including a light source that radiates monochromatic light onto a culture solution culturing erythrocytes accommodated in a culture container, a first photodetector that detects a light intensity of the monochromatic light transmitted through the culture solution, and a controller that evaluates an amount of hemoglobin in the culture solution based on a temporal change in the light intensity detected by the first photodetector.

According to this aspect, the monochromatic light is radiated from the light source onto the culture solution culturing the erythrocytes in the culture container, and the light intensity of the monochromatic light transmitted through the culture solution is detected by the first photodetector. In this case, because hemoglobin is the main substance constituting an erythrocyte, the amount of hemoglobin in the culture solution increases with increasing number of erythrocytes in the culture solution. The amount of monochromatic light transmitted through the culture solution, that is, the light intensity of the monochromatic light detected by the first photodetector, decreases with increasing amount of hemoglobin in the culture solution.

Therefore, the controller can readily determine whether or not the differentiation of the erythrocytes in the culture solution is properly progressing by evaluating the amount of hemoglobin in the culture solution based on a temporal change in the light intensity of the monochromatic light transmitted through the culture solution culturing the erythrocytes and detected by the first photodetector. Accordingly, the differentiation of the erythrocytes being cultured can be easily monitored.

In the above aspect, the controller may evaluate a degree of production of the hemoglobin based on a timing at which the light intensity stops changing.

When the production of hemoglobin in the culture solution is completed, the light intensity of the monochromatic light detected by the first photodetector stops decreasing. Therefore, the controller evaluates the degree of production of hemoglobin based on the timing at which the light intensity of the monochromatic light stops changing, so that the completion of production of hemoglobin in the culture solution can be readily ascertained.

In the above aspect, the controller may calculate a transmittance or an absorbance of the monochromatic light in the culture solution based on a ratio between a light intensity of the monochromatic light transmitted through the culture solution before culturing of the erythrocytes is started and a light intensity of the monochromatic light transmitted through the culture solution during the culturing of the erythrocytes, and may evaluate the amount of hemoglobin in the culture solution based on a temporal change in the calculated transmittance or absorbance.

Because the transmittance and absorbance of the monochromatic light in the culture solution change depending on the amount of hemoglobin in the culture solution, the degree of progression of differentiation of the erythrocytes can be readily determined in accordance with this configuration.

In the above aspect, the erythrocyte-culture monitoring device may further include a second photodetector that detects a light intensity of the monochromatic light before being transmitted through the culture solution. The controller may calculate a transmittance or an absorbance of the monochromatic light in the culture solution based on a ratio between the light intensity of the monochromatic light before being transmitted through the culture solution detected by the second photodetector and a light intensity of the monochromatic light after being transmitted through the culture solution detected by the first photodetector, and may evaluate the amount of hemoglobin in the culture solution based on a temporal change in the calculated transmittance or absorbance.

According to this configuration, the degree of progression of differentiation of the erythrocytes can be readily determined. With this configuration, even if the intensity of the monochromatic light emitted from the light source changes, the amount of hemoglobin in the culture solution can be properly evaluated. Accordingly, the degree of progression of differentiation of the erythrocytes can be accurately determined.

In the above aspect, the monochromatic light radiated onto the culture solution by the light source may be in a near-infrared wavelength band between an absorption wavelength band of water and an absorption wavelength band of the culture solution.

By using monochromatic light in a wavelength band that excludes the absorption wavelength band of water and the absorption wavelength band of the culture solution, the effect of absorption of monochromatic light by water and culture solution can be reduced. Accordingly, the amount of hemoglobin in the culture solution can be accurately evaluated.

In the above aspect, the near-infrared wavelength band may be between 700 nm and 900 nm.

When the absorption coefficient of hemoglobin is large and the density of hemoglobin is high, the amount of monochromatic light detected by the first photodetector decreases, resulting in a lower S/N ratio. Because the hemoglobin absorption coefficient is relatively small in the wavelength band between 700 nm and 900 nm, the amount of hemoglobin in the culture solution can be evaluated more accurately in accordance with this configuration.

In the above aspect, the near-infrared wavelength band may be a range in which an absorption coefficient of oxygenated hemoglobin and an absorption coefficient of deoxygenated hemoglobin are substantially equal to each other.

The absorption spectra of oxygenated hemoglobin (Oxy Hb) and deoxygenated hemoglobin (Deoxy Hb) differ significantly. Therefore, the measurement of the light intensity of monochromatic light becomes unstable due to oxygen concentration, sometimes making it impossible to accurately determine the timing at which the amount of monochromatic light transmitted through the culture solution becomes saturated. By setting the wavelength band of near-infrared light to be radiated onto the culture solution to a range in which the absorption coefficient of oxygenated hemoglobin and the absorption coefficient of deoxygenated hemoglobin are substantially equal to each other, the amount of transmitted light does not change between oxygenated hemoglobin and deoxygenated hemoglobin. Thus, the timing at which the amount of monochromatic light transmitted through the culture solution becomes saturated can be accurately determined.

In the above aspect, the light source may vary a wavelength of the monochromatic light for measuring the light intensity between a cell proliferating process for causing cells serving as a base for the erythrocytes to proliferate and a hemoglobin increasing process for causing the cells proliferated as a result of the cell proliferating process to change into the hemoglobin.

With regard to a change in the light intensity of monochromatic light transmitted through the culture solution, scattering of the monochromatic light caused by the cells is dominant in the cell proliferating process, whereas the amount of hemoglobin in the culture solution is dominant in the hemoglobin increasing process. Therefore, by varying the wavelength of the monochromatic light between the cell proliferating process and the hemoglobin increasing process, the degree of progression of culturing can be accurately ascertained for each process.

For example, since the monochromatic light used in the cell proliferating process has a wavelength that tends to cause scattering, the degree of proliferation of the cells in the culture solution can be detected with high accuracy. Since the monochromatic light used in the hemoglobin increasing process has a wavelength with a high hemoglobin transmittance, an S/N ratio for measuring the light intensity of the monochromatic light transmitted through the culture solution can be ensured.

In the above aspect, the light source and the first photodetector may be disposed outside the culture container, the light source may radiate the monochromatic light from outside the culture container toward inside the culture container, and the first photodetector may detect the monochromatic light output outside the culture container after being transmitted through the culture solution.

With this configuration, a simple configuration can be achieved in the culture container.

In the above aspect, the erythrocyte-culture monitoring device may further include a retroreflector member and an optical-path splitting member. The retroreflector member reflects the monochromatic light output outside the culture container and causes the monochromatic light to return toward the culture container via the same optical path as an optical path of the monochromatic light entering the retroreflector member. The optical-path splitting member splits an optical path of the monochromatic light reflected by the retroreflector member and subsequently transmitted again through the culture container, and guides the split monochromatic light toward the first photodetector.

According to this configuration, the retroreflector member and the optical-path splitting member can cause the monochromatic light transmitted through the culture solution to enter the first photodetector, regardless of the surface shape, size, and position of the culture container. Therefore, the light intensity of the monochromatic light transmitted through the culture solution can be accurately measured even when various types of culture containers are used.

In the above aspect, one of the light source and the first photodetector may be disposed inside the culture container, and the other one of the light source and the first photodetector may be disposed outside the culture container. The first photodetector disposed inside the culture container may detect the monochromatic light radiated from the light source disposed outside the culture container and transmitted through the culture solution, or the first photodetector disposed outside the culture container may detect the monochromatic light radiated from the light source disposed inside the culture container and transmitted through the culture solution.

According to this configuration, since one of the light source and the first photodetector is disposed inside the culture container, space can be ensured outside the culture container.

### REFERENCE SIGNS LIST

1, 31, 41 erythrocyte-culture monitoring device
7 controller
11 culture container
19 light source
21 photodetector (first photodetector)
22b photodetector (second photodetector)
33 retroreflector member
22a, 35 half mirror (optical-path splitting member)
S cell

1. An erythrocyte-culture monitoring device comprising:
a light source that is configured to radiate monochromatic light onto a culture solution culturing erythrocytes accommodated in a culture container;
a first photodetector that is configured to detect a light intensity of the monochromatic light transmitted through the culture solution; and
a controller that is configured to evaluate an amount of hemoglobin in the culture solution based on a temporal change in the light intensity detected by the photodetector.

2. The erythrocyte-culture monitoring device according to claim 1,
wherein the controller evaluates a degree of production of the hemoglobin based on a timing at which the light intensity stops changing.

3. The erythrocyte-culture monitoring device according to claim 1,
wherein the controller calculates a transmittance or an absorbance of the monochromatic light in the culture solution based on a ratio between a light intensity of the monochromatic light transmitted through the culture solution before culturing of the erythrocytes is started and a light intensity of the monochromatic light transmitted through the culture solution during the culturing of the erythrocytes, and evaluates the amount of hemoglobin in the culture solution based on a temporal change in the calculated transmittance or absorbance.

4. The erythrocyte-culture monitoring device according to claim 1, further comprising:
a second photodetector that is configured to detect a light intensity of the monochromatic light before being transmitted through the culture solution,
wherein the controller calculates a transmittance or an absorbance of the monochromatic light in the culture solution based on a ratio between the light intensity of the monochromatic light before being transmitted through the culture solution detected by the second photodetector and a light intensity of the monochromatic light after being transmitted through the culture solution detected by the first photodetector, and evaluates the amount of hemoglobin in the culture solution based on a temporal change in the calculated transmittance or absorbance.

5. The erythrocyte-culture monitoring device according to claim 1,
wherein the monochromatic light radiated onto the culture solution by the light source is in a near-infrared wavelength band between an absorption wavelength band of water and an absorption wavelength band of the culture solution.

6. The erythrocyte-culture monitoring device according to claim 5,
wherein the near-infrared wavelength band is between 700 nm and 900 nm.

7. The erythrocyte-culture monitoring device according to claim 5,
wherein the near-infrared wavelength band is a range in which an absorption coefficient of oxygenated hemoglobin and an absorption coefficient of deoxygenated hemoglobin are substantially equal to each other.

8. The erythrocyte-culture monitoring device according to claim 1,
wherein the light source varies a wavelength of the monochromatic light for measuring the light intensity between a cell proliferating process for causing cells serving as a base for the erythrocytes to proliferate and a hemoglobin increasing process for causing the cells proliferated as a result of the cell proliferating process to change into the hemoglobin.

9. The erythrocyte-culture monitoring device according to claim 1,
wherein the light source and the first photodetector are disposed outside the culture container,
wherein the light source radiates the monochromatic light from outside the culture container toward inside the culture container, and
wherein the first photodetector detects the monochromatic light output outside the culture container after being transmitted through the culture solution.

10. The erythrocyte-culture monitoring device according to claim 9, further comprising:
a retroreflector member that is configured to reflect the monochromatic light output outside the culture container and causes the monochromatic light to return toward the culture container via the same optical path as an optical path of the monochromatic light entering the retroreflector member; and
an optical-path splitting member that is configured to split an optical path of the monochromatic light reflected by the retroreflector member and subsequently transmitted again through the culture container, and guides the split monochromatic light toward the first photodetector.

11. The erythrocyte-culture monitoring device according to claim 1,
wherein one of the light source and the first photodetector is disposed inside the culture container, and the other one of the light source and the first photodetector is disposed outside the culture container, and
wherein the first photodetector disposed inside the culture container detects the monochromatic light radiated from the light source disposed outside the culture container and transmitted through the culture solution, or
wherein the first photodetector disposed outside the culture container detects the monochromatic light radiated from the light source disposed inside the culture container and transmitted through the culture solution.

12. The erythrocyte-culture monitoring device according to claim 2,
wherein the monochromatic light radiated onto the culture solution by the light source is in a near-infrared wavelength band between an absorption wavelength band of water and an absorption wavelength band of the culture solution.

13. The erythrocyte-culture monitoring device according to claim 12,
wherein the near-infrared wavelength band is between 700 nm and 900 nm.

14. The erythrocyte-culture monitoring device according to claim 12,
wherein the near-infrared wavelength band is a range in which an absorption coefficient of oxygenated hemoglobin and an absorption coefficient of deoxygenated hemoglobin are substantially equal to each other.

15. The erythrocyte-culture monitoring device according to claim 2,
wherein the light source varies a wavelength of the monochromatic light for measuring the light intensity between a cell proliferating process for causing cells serving as a base for the erythrocytes to proliferate and a hemoglobin increasing process for causing the cells proliferated as a result of the cell proliferating process to change into the hemoglobin.

16. The erythrocyte-culture monitoring device according to claim 2,
wherein the light source and the first photodetector are disposed outside the culture container,
wherein the light source radiates the monochromatic light from outside the culture container toward inside the culture container, and
wherein the first photodetector detects the monochromatic light output outside the culture container after being transmitted through the culture solution.

17. The erythrocyte-culture monitoring device according to claim 16, further comprising:
a retroreflector member that is configured to reflect the monochromatic light output outside the culture container and causes the monochromatic light to return toward the culture container via the same optical path as an optical path of the monochromatic light entering the retroreflector member; and
an optical-path splitting member that is configured to split an optical path of the monochromatic light reflected by the retroreflector member and subsequently transmitted again through the culture container, and guides the split monochromatic light toward the first photodetector.

18. The erythrocyte-culture monitoring device according to claim 2,
wherein one of the light source and the first photodetector is disposed inside the culture container, and the other one of the light source and the first photodetector is disposed outside the culture container, and
wherein the first photodetector disposed inside the culture container detects the monochromatic light radiated from the light source disposed outside the culture container and transmitted through the culture solution, or
wherein the first photodetector disposed outside the culture container detects the monochromatic light radiated from the light source disposed inside the culture container and transmitted through the culture solution.
